(19) United States
(12) Patent Application Publication
(10) Pub. No.: US 2025/0290031 A1
(43) Pub. Date: Sep. 18, 2025

(54) Phage-Resistant Lactic Acid Bacterium

(71) Applicant: CHR. HANSEN A/S, Hoersholm (DK)

(72) Inventors: Thomas JANZEN, Hoersholm (DK); Ditte Ellegaard CHRISTIANSEN, Hoersholm (DK)

(73) Assignee: CHR. HANSEN A/S, Hoersholm (DK)

(21) Appl. No.: 18/859,853

(22) PCT Filed: May 15, 2023
(86) PCT No.: PCT/EP2023/062914

§ 371 Date: Oct. 24, 2024

(30) Foreign Application Priority Data
May 17, 2022 (EP) 22173799.2

(51) Int. Cl. C12N 1/20; A23C 9/123; C12R 1/225; C12R 1/46

(52) U.S. Cl. CPC C12N 1/205; A23C 9/1238; A23V 2400/123; A23V 2400/249; C12R 2001/225; C12R 2001/46

(57) ABSTRACT

The present invention relates to a phage-resistant lactic acid bacterium, compositions comprising the bacterium, and fermented food products obtained with the bacterium or compositions comprising the bacterium.

### FIELD OF INVENTION

The present invention relates to a phage-resistant lactic acid bacterium, compositions comprising the bacterium, and fermented food products manufactured with the bacterium.

### BACKGROUND OF INVENTION

The food industry uses numerous bacteria, in particular lactic acid bacteria in the production of fermented food. The choice of lactic acid bacteria impacts the feature of the fermented food product, such as e.g. taste, texture, and shelf life.

Production failures due to bacteriophage infection of the bacteria are considered to be one of the major problems in industrial use of bacterial cultures. Bacteriophages have been found for many of the bacterial strains used in the industry, such as species of lactic acid bacteria e.g. Lactococcus sp., Lactobacillus sp., Leuconostoc sp., Pediococcus sp. or Streptococcus sp.

In the food industry lactic acid bacterial starter cultures are widely used for food fermentations. A factor, which leads to frequent bacteriophage infections in lactic acid bacterial starter cultures, is the fact that the fermentation conditions in the food industry including the dairy industry are generally non-sterile. Thus, it has not yet been possible to eliminate bacteriophage contamination under these industrial conditions.

The lytic development of bacteriophages involves adsorption of the phages to the host cell surface, injection of phage DNA into the cell, synthesis of phage proteins, replication of phage DNA, assembly of progeny phages and release of progeny from the host. Cell-mediated mechanisms of interference with any of these events can prevent a phage infection. The ability of bacterial cultures to resist bacteriophage infection during industrial use depends to a large extent on host strain characteristics affecting one or more of the above mechanisms.

In order to meet the requirements of the industry, it has become necessary to propose novel phage-resistant strains of lactic acid bacteria, in particular of Streptococcus thermophilus.

### SUMMARY OF INVENTION

In a first aspect the present invention relates to a lactic acid bacterium with increased phage-resistance towards DSM 34256 and/or DSM 34257 as compared to its mother strain.

In a second aspect, the invention relates to a composition comprising the lactic acid bacteria of the invention.

In a third aspect, the invention relates to a method for producing a fermented food product, comprising fermenting a substrate with the lactic acid bacterium of the invention, or a composition of the invention.

In a fourth aspect, the invention relates to a fermented food product comprising the lactic acid bacterium of the invention or obtainable by the method of the invention.

### DETAILED DESCRIPTION OF THE INVENTION

Prior to outlining the present invention in more details, a set of terms and conventions is defined:

The term “genus” means genus as defined on the website: www.ncbi.nlm.nih.gov/taxonomy. A bacterial “strain” as used herein refers to a bacterium which remains genetically unchanged when grown or multiplied. A multiplicity of identical bacteria is included.

In the present context, the term “mutant” or “mutant strain” should be understood as a strain derived, or a strain which can be derived, from a strain of the invention (or the mother strain) by means of e.g. genetic engineering, radiation and/or chemical treatment. It is preferred that the mutant is a functionally equivalent mutant, e.g. a mutant that has substantially the same, or improved, properties (e.g. regarding texture, shear stress, viscosity, gel firmness, mouth coating, flavor, post acidification, acidification speed, and/or phage robustness) as the strain from which it is derived. Such a mutant is a part of the present invention. Especially, the term “mutant” refers to a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light, or to a spontaneously occurring mutant. A mutant may have been subjected to several mutagenization treatments (a single treatment should be understood one mutagenization step followed by a screening/selection step), but it is presently preferred that no more than 20, or no more than 10, or no more than 5, treatments (or screening/selection steps) are carried out. In a presently preferred mutant, less than 5%, or less than 1% or even less than 0.1% of the nucleotides in the bacterial genome have been shifted with another nucleotide, or deleted, compared to the mother strain. As will be clear to the skilled person mutants of the present invention can also be mother strains.

In the present context, the term “variant” or “variant strain” should be understood as a strain which is functionally equivalent to a strain of the invention, e.g. having substantially the same, or improved, properties or characteristics e.g. texture, acidification speed, viscosity, gel firmness, mouth coating, flavor, post acidification and/or phage robustness). Such variants, which may be identified using appropriate screening techniques, are a part of the present invention.

In connection with strains of the genus Lactobacillus, the term “CFU” means colony forming units as determined by growth (forming a colony) on an MRS agar plate incubated at anaerobic conditions at 37° C. for 3 days. The MRS agar has the following composition (g/l):
Bacto Proteose Peptone No. 3: 10.0
Bacto Beef extract: 10.0
Bacto Yeast extract: 5.0
Dextrose: 20.0
Sorbitan Monooleate Complex: 1.0
Ammonium Citrate: 2.0
Sodium Acetate: 5.0
Magnesium Sulfate: 0.1
Manganese Sulfate: 0.05
Potassium Phosphate Dibasis: 2.0
Bacto Agar: 15.0
Milli-Q water: 1000 ml.

pH is adjusted to 5.4 or 6.5: pH is adjusted to 6.5 for L. rhamnosus, L. casei and L. paracasei. For all other Lactobacillus species the pH is adjusted to 5.4. In particular, pH is adjusted to 5.4 for L. delbrueckii subsp. bulgaricus; L. acidophilus and L. helveticus. pH is adjusted to 6.5 for L. rhamnosus, L. casei and L. paracasei.

In connection with S. thermophilus, the term “CFU” means colony forming units as determined by growth (forming a colony) on an M 17 agar plate incubated at aerobic conditions at 37° C. for 3 days. The M 17 agar has the following composition (g/l):
Tryptone: 2.5 g
Peptic digest of meat: 2.5 g
Papaic digest of soybean meal: 5.0 g
Yeast extract: 2.5 g
Meat extract: 5.0 g
Lactose: 5.0 g
Sodium-glycero-phosphate: 19.0 g
Magnesium sulphate, 7 H20:0.25 g
Ascorbic acid: 0.5 g
Agar: 15.0 g
Milli-Q water: 1000 ml.
pH is adjusted to final pH 7.1±0.2 (25° C.)

### Phage-Resistant Lactic Acid Bacteria

Phage-resistant mutant strains may be generated by exposing a phage-sensitive mother strain to the desired phage or phages, e.g. by growing the phage-sensitive mother strain in the presence of a concentration of the desired phage(s) sufficient to induce a selection pressure on said strain. Methods of generating phage-resistant mutants have previously been described. Phage-resistant mutants of present the invention may be obtained by the method as described in the examples. In one aspect the present invention relates to a lactic acid bacterium with increased phage resistance towards DSM 34256 and/or DSM 34257 as compared to its mother strain.

It may furthermore be desirable to increase the texture provided by such a strain. In that case a selection of phage-resistant mutants with increased texturizing properties should be applied. Phage-resistant mutants of present the invention with increased texturizing property such as viscosity may be obtained by the method as described in the examples. In one embodiment the invention relates to the lactic acid bacterium of the invention, wherein the bacterium has increased viscosity as compared to its mother strain.

It will be appreciated that phage-resistance may be obtained by the method with strains originating from many species. However, in the present invention a preferred species is Streptococcus thermophilus. In one embodiment the invention relates to the lactic acid bacterium of the invention, wherein the bacterium belongs to the species Streptococcus thermophilus.

Any Streptococcus thermophilus strain may be used as a mother strain. The mother strain of the present invention is preferably the Streptococcus thermophilus strain DSM 34236 or a mutant or variant thereof. In one embodiment the invention relates to the lactic acid bacterium of the invention, wherein the mother strain is DSM 34236.

The preferred mutant derived from the mother strain may be any mutant with improved phage-resistance and optionally increased viscosity as compared to its mother strain. In one embodiment the invention relates to the lactic acid bacterium of the invention, wherein the bacterium is selected from the group consisting of: DSM 34235, and mutants and variants thereof.

As used herein, the term “lactic acid bacterium” abbreviated “LAB” designates a gram-positive, microaerophilic or anaerobic bacterium, which ferments sugars with the production of acids including lactic acid as the predominantly produced acid, acetic acid and propionic acid. The industrially most useful lactic acid bacteria are found within the order “Lactobacillies” which includes Lactococcus spp., Streptococcus spp., Lactobacillus spp., Leuconostoc spp., Pseudoleuconostoc spp., Pediococcus spp., Brevibacterium spp., Enterococcus spp. and Propionibacterium spp. Additionally, lactic acid producing bacteria belonging to the group of the strict anaerobic bacteria, bifidobacteria, i.e. Bifidobacterium spp., are generally included in the group of lactic acid bacteria. These are frequently used as food cultures alone or in combination with other lactic acid bacteria. Lactic acid bacteria, including bacteria of the species Lactobacillus sp. and Streptococcus thermophilus, are normally supplied to the dairy industry either as frozen or freeze-dried cultures for bulk starter propagation or as so-called “Direct Vat Set” (DVS) cultures, intended for direct inoculation into a fermentation vessel or vat for the production of a dairy product, such as a fermented milk product. Such cultures are in general referred to as “starter cultures” or “starters”.

The term “improved resistance to a bacteriophage” or “improved phage-resistance” denotes that the bacteria strain when tested in a plaque assay, such as the assay “determination of phage resistance by the agar overplay method” described in the examples have an improved phage resistance to at least one phage expressed as the difference in pfu/mL (plaque forming unit per mL) obtainable with said at least one bacteriophage on the given strain, compared to the pfu/mL obtainable with the same bacteriophage on the parent strain. A strain with improved resistance to a bacteriophage preferably show a reduction of pfu/mL of a factor of at least 50, of at least 100, of at least 500, of at least 1000, of at least 5000, of at least 10000 or more.

### Compositions

The lactic acid bacterium of the invention as described herein is useful in compositions, more particular in starter cultures for preparing fermented food or feed products. In one aspect the invention relates to a composition comprising the lactic acid bacteria of the invention.

As it is normal in the production of lactic acid bacterial fermentation processes to apply mixed cultures of lactic acid bacteria, the composition will in some embodiments comprise a multiplicity of strains either belonging to the same species or belonging to different species.

In one embodiment the composition of the invention, further comprising, either as a mixture or as a kit-of-parts one or more strains belonging to a Lactobacillus species.

In one embodiment, the one or more strains belonging to the genus Lactobacillus may be selected from a group consisting of: L. delbrueckii, L. delbrueckii subsp bulgaricus, L. delbrueckii subsp. lactis.

In one embodiment the Lactobacillus bacteria strain of the invention is L. bulgaricus. L. bulgaricus is a lactic acid bacterium which is frequently employed for commercial milk fermentation where the organism is normally used as part of a mixed starter culture. In one embodiment the composition of the invention, wherein at least one Lactobacillus strain is Lactobacillus delbrueckii subsp. bulgaricus.

In one embodiment the composition of the invention, wherein the Lactobacillus bulgaricus strain is selected from the group consisting of: DSM 19251; DSM 19252; DSM 22586; DSM 26419; DSM 28910; DSM 32092; DSM 33515; and mutants and variants thereof.

In one embodiment the composition and/or mixture or kit-of-parts comprises S. thermophilus strain DSM 34235 or mutants or variants thereof, and a strain belonging to the species Lactobacillus. Thus, in a preferred embodiment the composition and/or mixture or kit-of-parts comprises S. thermophilus strain DSM 34235 or mutants or variants thereof, and a strain belonging to the species Lactobacillus delbrueckii subsp. bulgaricus. Thus, in a preferred embodiment the composition and/or mixture or kit-of-parts comprises S. thermophilus strain DSM 34235 or mutants or variants thereof, and a strain belonging to the species Lactobacillus bulgaricus strain selected from the group consisting of: DSM 19251; DSM 19252; DSM 22586; DSM 26419; DSM 28910; DSM 32092; DSM 33515; and mutants and variants thereof.

In addition to the lactic acid bacteria of the invention the composition may comprise further strains of Streptococcus thermophilus. In one embodiment the composition further comprises either as a mixture or as a kit-of-parts one or more Streptococcus thermophilus strains. In one embodiment the composition of the invention, comprising either as a mixture or as a kit-of-parts one or more Streptococcus thermophilus strains selected from the group consisting of: DSM 17876; DSM 18111; DSM 19242; DSM 21408; DSM 22585; DSM 22587; DSM 22588; DSM 22589; DSM 22935; DSM 24023; DSM 25012; DSM 26562; DSM 32503; DSM 32826; DSM 33869; and mutants and variants thereof.

In one embodiment the composition and/or mixture or kit-of-parts comprises Streptococcus thermophilus strain DSM 34235 or mutants or variants thereof, and a strain belonging to the species Streptococcus. Thus, in a preferred embodiment the composition and/or mixture or kit-of-parts comprises Streptococcus thermophilus strain DSM 34235 or mutants or variants thereof, and a strain belonging to the species Streptococcus thermophilus. Thus, in a preferred embodiment the composition and/or mixture or kit-of-parts comprises Streptococcus thermophilus strain DSM 34235 or mutants or variants thereof, and a strain belonging to the species Streptococcus thermophilus strain selected from the group consisting of: DSM 17876; DSM 18111; DSM 19242; DSM 21408; DSM 22585; DSM 22587; DSM 22588; DSM 22589; DSM 22935; DSM 24023; DSM 25012; DSM 26562; DSM 32503; DSM 32826; DSM 33869; and mutants and variants thereof.

In one embodiment the composition of the invention, further comprising, either as a mixture or as a kit-of-parts one or more strains belonging to a Lactococcus species. In one embodiment, the one or more strains belonging to the genus Lactococcus may be selected from a group consisting of: L. lactis; L. lactis subsp lactis; L. lactis subsp. cremoris. In one embodiment the composition of the invention, further comprising either as a mixture or as a kit-of-parts one or more Lactococcus strains selected from the group consisting of: DSM 21404; DSM 33192; DSM 21405; and mutants and variants thereof.

In one embodiment the composition of the invention, further comprising, either as a mixture or as a kit-of-parts one or more strains belonging to a Pediococcus species.

In one embodiment, the one or more strains belonging to the genus Pediococcus may be a Pediococcus acidilactis strain. In one embodiment the composition of the invention, further comprising either as a mixture or as a kit-of-parts one or more Pediococcus acidilactis strain selected from the group consisting of: DSM 28307; and mutants and variants thereof.

The composition of the present invention may comprise probiotic bacteria. Probiotic bacterial strains may be added before or after fermentation. If added before fermentation the probiotic bacterial strain also acts as fermentative bacteria. The term “probiotic bacteria” refers to viable bacteria which are administered in adequate amounts to a consumer for the purpose of achieving a health-promoting effect in the consumer. Probiotic bacteria are capable of surviving the conditions of the gastrointestinal tract after ingestion and colonize the intestine of the consumer.

It will be appreciated that the Lactobacillus genus taxonomy was updated in 2020. The new taxonomy is disclosed in Zheng et al. 2020 and will be cohered to herein if not otherwise indicated. For the purpose of the present invention, the table below presents a list of new and old names of some Lactobacillus species relevant to the present invention.

TABLE 1
New and old names of some Lactobacillus
species relevant to the present invention.

| OLD NAME | NEW NAME |
|---|---|
| Lactobacillus reuteri | Limosilactobacillus reuteri |
| Lactobacillus rhamnosus | Lacticaseibacillus rhamnosus |
| Lactobacillus salivarius | Ligilactobacillus salivarius |
| Lactobacillus casei | Lacticaseibacillus casei |
| Lactobacillus paracasei | Lacticaseibacillus paracasei |
| subsp. paracasei | subsp. Paracasei |
| Lactobacillus plantarum | Lactiplantibacillus plantarum |
| subsp. plantarum | subsp. plantarum |
| Lactobacillus fermentum | Limosilactobacillus fermentum |
| Lactobacillus animalis | Ligilactobacillus animalis |
| Lactobacillus buchneri | Lentilactobacillus buchneri |
| Lactobacillus curvatus | Latilactobacillus curvatus |
| Lactobacillus futsaii | Companilactobacillus futsaii |
| Lactobacillus sakei subsp. sakei | Latilactobacillus sakei subsp. |
| Lactobacillus pentosus | Lactiplantibacillus pentosus |

In one embodiment of the present invention the probiotic strain is selected from the group consisting of bacteria of the genus Lactobacillus, such as Lactobacillus acidophilus, Lacticaseibacillus paracasei, Lacticaseibacillus rhamnosus, Lacticaseibacillus casei, Lactobacillus delbrueckii, Limosilactobacillus fermentum, Lactobacillus lactis, Lactiplantibacillus plantarum, Limosilactobacillus reuteri and Lactobacillus johnsonii, the genus Bifidobacterium, such as the Bifidobacterium longum, Bifidobacterium adolescentis, Bifidobacterium bifidum, Bifidobacterium breve, Bifidobacterium animalis subsp. lactis, Bifidobacterium dentium, Bifidobacterium catenulatum, Bifidobacterium angulatum, Bifidobacterium magnum, Bifidobacterium pseudocatenulatum and Bifidobacterium infantis, and the like.

In one embodiment of the invention, the probiotic Lactobacillus strain is selected from the group consisting of Lactobacillus acidophilus, Lacticaseibacillus paracasei, Lacticaseibacillus rhamnosus, Lacticaseibacillus casei, Lactobacillus delbrueckii, Lactobacillus lactis, Lactiplantibacillus plantarum, Limosilactobacillus reuteri and Lactobacillus johnsonii. In one embodiment of the invention, the probiotic strain is Lactobacillus acidophilus (LA-5®) deposited as DSM 13241.

In one embodiment of the invention, the probiotic Lactobacillus strain is selected from the group consisting of a Lacticaseibacillus rhamnosus strain, a Lacticaseibacillus paracasei strain and a Limosilactobacillus fermentum strain. In one embodiment of the invention, the probiotic strain is Lacticaseibacillus rhamnosus strain DSM 33870 or LGG® deposited as ATCC 53103. In one embodiment of the invention, the probiotic strain is Lacticaseibacillus paracasei strain DSM 19465 or CRL 431 deposited as ATCC 55544. In one embodiment of the invention, the probiotic strain is Limosilactobacillus fermentum strain DSM 22584.

In one embodiment of the invention, the probiotic Bifidobacterium strain is selected from the group consisting of Bifidobacterium longum, Bifidobacterium adolescentis, Bifidobacterium bifidum, Bifidobacterium breve, Bifidobacterium animalis subsp. lactis, Bifidobacterium dentium, Bifidobacterium catenulatum, Bifidobacterium angulatum, Bifidobacterium magnum, Bifidobacterium pseudocatenulatum and Bifidobacterium infantis. In one embodiment of the invention, the probiotic Bifidobacterium strain is Bifidobacterium animalis subsp. lactis. In one embodiment of the invention the Bifidobacterium animalis subsp. lactis strain is DSM 33868 or BB-12® deposited as DSM 15954.

The above mixtures or kit-of-parts may be further combined with other lactic acid bacteria such as but not limited to probiotic bacteria. In one embodiment the one or more lactic acid bacteria is selected from the group consisting of Bifidobacterium such as Bifidobacterium animalis subsp. lactis (e.g. BB-12®), Lactobacillus acidophilus (e.g. LA-5®), Lacticaseibacillus rhamnosus (e.g. LGG®) and any combinations thereof. Which Bifidobacterium, Lactobacillus acidophilus and/or Lacticaseibacillus rhamnosus to apply depend on their application and food to be produced.

The expression “A kit-of-part” comprising strain(s) means that strains or culture of strain(s) are physically separated but intended to be used together. Thus, the strains or culture of S. thermophilus strain(s) and Lactobacillus strain(s) are in different boxes or sachets. In one embodiment, the S. thermophilus strain(s) and the Lactobacillus such as e.g. Lactobacillus delbrueckii subsp bulgaricus, Lactobacillus acidophilus, Lacticaseibacillus casei, Lacticaseibacillus paracasei, and/or Limosilactobacillus reuteri strain(s) are under the same format, i.e., are in a frozen format, in the form of pellets or frozen pellets, a powder form, such as a dried or freeze-dried powder.

In one embodiment the invention relates to the composition of the invention, comprising either as a mixture or as a kit-of-parts one or more probiotic strains selected from Bifidobacterium, Lactobacillus acidophilus, Lacticaseibacillus casei (previous Lactobacillus casei), Lacticaseibacillus paracasei (previous Lactobacillus paracasei), Lacticaseibacillus rhamnosus (previous Lactobacillus rhamnosus), and Limosilactobacillus fermentum (previous lactobacillus fermentum).

In one embodiment the composition of the invention comprises bacteria in a concentrated form where the total concentration of viable cells is in the range of 10⁴ to 10¹² cfu (colony forming units) per gram of the composition including at least 10⁴ cfu per gram of the composition, such as at least 10⁵ cfu/g, e.g. at least 10⁶ cfu/g, such as at least 10⁷ cfu/g, e.g. at least 10⁸ cfu/g, such as at least 10⁹ cfu/g, e.g. at least 10¹⁰ cfu/g, such as at least 10¹¹ cfu/g.

In one embodiment the composition of the invention comprises bacteria in a concentrated form where the concentration of each strain of viable cells is in the range of 10⁴ to 10¹² cfu (colony forming units) per gram of the composition including at least 10⁴ cfu per gram of the composition, such as at least 10⁵ cfu/g, e.g. at least 10⁶ cfu/g, such as at least 10⁷ cfu/g, e.g. at least 10⁸ cfu/g, such as at least 10⁹ cfu/g, e.g. at least 10¹⁰ cfu/g, such as at least 10¹¹ cfu/g.

In one embodiment of the present invention, the composition comprises from 10⁴ to 10¹² CFU (colony forming units)/g of the S. thermophilus strain, from 10⁵ to 10¹¹ CFU/g, from 10⁶ to 10¹⁰ CFU/g, or from 10⁷ to 10⁹ CFU/g of the S. thermophilus strain.

In one embodiment the composition further comprises from 10⁴ to 10¹² CFU/g of the Lactobacillus strain, from 10⁵ to 10¹¹ CFU/g, from 10⁶ to 10¹⁰ CFU/g, or from 10⁷ to 10⁹ CFU/g of the Lactobacillus strain.

In one embodiment the composition comprises from 10⁴ to 10¹² CFU/g, from 10⁵ to 10¹¹ CFU/g, from 10⁶ to 10¹⁰ CFU/g, or from 10⁷ to 10⁹ CFU/g of each of the Lactobacillus delbrueckii subsp Lactobacillus bulgaricus, acidophilus, Lacticaseibacillus casei, Lacticaseibacillus paracasei, and/or Limosilactobacillus reuteri strain(s).

S. thermophilus, Lactobacillus and Lacticaseibacillus such as L. bulgaricus, L. acidophilus, L. casei, L. paracasei, and/or L. rhamnosus and other lactic acid bacteria are commonly used as starter cultures serving a technological purpose in the production of various foods, such as in the dairy industry, such as for fermented milk products. Thus, in another preferred embodiment the composition is a starter culture. The composition may be a starter culture such as a yoghurt starter culture.

The composition and/or starter culture may be frozen, spray-dried, freeze-dried, vacuum-dried, air dried, tray dried or in liquid form. Typically, the storage stability of the composition and/or starter culture can be extended by formulating the product with low water activity. By controlling the water activity (Aw), it is possible to predict and regulate the effect of moisture migration on the product. Therefore, it may be preferred that the water activity (Aw) of the dried compositions herein is in the range from 0.01-0.8, preferably in the range from 0.05-0.4.

The composition may comprise further components such as e.g. cryoprotectants and/or conventional additives including nutrients such as yeast extracts, sugars and vitamins.

The composition of the present invention may be provided in several forms. It may be a powder, pellets or tablets. It may be a frozen form, dried form, freeze dried form, or liquid form. Thus, in one embodiment the composition is in frozen, dried, freeze-dried or liquid form. In one embodiment the composition of the invention, wherein said composition is in frozen, freeze-dried or liquid form.

The composition of the present invention may additionally comprise cryoprotectants, lyoprotectants, antioxidants, nutrients, fillers, flavorants or mixtures thereof. The composition preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as cryoprotectants and lyoprotectantare known to a skilled person in the art. Suitable cryoprotectants or lyoprotectants include mono-, di-, tri- and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate).

In one embodiment, the composition according to the present invention may comprise one or more cryoprotective agent(s) selected from the group consisting of inosine-5′-monophosphate (IMP), adenosine-5′-monophosphate (AMP), guanosine-5′-monophosphate (GMP), uranosine-5′-monophosphate (UMP), cytidine-5′-monophosphate (CMP), adenine, guanine, uracil, cytosine, adenosine, guanosine, uridine, cytidine, hypoxanthine, xanthine, hypoxanthine, orotidine, thymidine, inosine and a derivative of any such compounds. Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorants.

In one embodiment of the invention the cryoprotective agent is an agent or mixture of agents, which in addition to its cryoprotectivity has a booster effect. The expression “booster effect” is used to describe the situation wherein the cryoprotective agent confers an increased metabolic activity (booster effect) on to the thawed or reconstituted culture when it is inoculated into the medium to be fermented or converted. Viability and metabolic activity are not synonymous concepts. Commercial frozen or freeze-dried cultures may retain their viability, although they may have lost a significant portion of their metabolic activity e.g. cultures may lose their acid-producing (acidification) activity when kept stored even for shorter periods of time. Thus, viability and booster effect have to be evaluated by different assays. Whereas viability is assessed by viability assays such as the determination of colony forming units, booster effect is assessed by quantifying the relevant metabolic activity of the thawed or reconstituted culture relative to the viability of the culture. The term “metabolic activity” refers to the oxygen removal activity of the cultures, its acid-producing activity, i. e. the production of e. g. lactic acid, acetic acid, formic acid and/or propionic acid, or its metabolite producing activity such as the production of aroma compounds such as acetaldehyde, (a-acetolactate, acetoin, diacetyl and 2,3-butylene glycol (butanediol)).

In one embodiment the composition of the invention contains or comprises from 0.2% to 20% of the cryoprotective agent or mixture of agents measured as % w/w of the material. It is, however, preferable to add the cryoprotective agent or mixture of agents at an amount which is in the range from 0.2% to 15%, from 0.2% to 10%, from 0.5% to 7%, and from 1% to 6% by weight, including within the range from 2% to 5% of the cryoprotective agent or mixture of agents measured as % w/w of the frozen material by weight. In a preferred embodiment the culture comprises approximately 3% of the cryoprotective agent or mixture of agents measured as % w/w of the material by weight. The amount of approximately 3% of the cryoprotective agent corresponds to concentrations in the 100 mM range. It should be recognized that for each aspect of embodiment of the invention the ranges may be increments of the described ranges.

In the present context the term “from x % to y %” means to include the end-points, thus equal to the term “from and including x % to and including y %”

In a further aspect, the composition of the present invention contains or comprises an ammonium salt (e.g. an ammonium salt of organic acid (such as ammonium formate and ammonium citrate) or an ammonium salt of an inorganic acid) as a booster (e.g. growth booster or acidification booster) for bacterial cells, such as cells belonging to the species S. thermophilus, e.g. (substantial) urease negative bacterial cells. The term “ammonium salt”, “ammonium formate”, etc., should be understood as a source of the salt or a combination of the ions. The term “source” of e.g. “ammonium formate” or “ammonium salt” refers to a compound or mix of compounds that when added to a culture of cells, provides ammonium formate or an ammonium salt. In some embodiments, the source of ammonium releases ammonium into a growth medium, while in other embodiments, the ammonium source is metabolized to produce ammonium. In some preferred embodiments, the ammonium source is exogenous. In some particularly preferred embodiments, ammonium is not provided by the dairy substrate. It should of course be understood that ammonia may be added instead of ammonium salt. Thus, the term ammonium salt comprises ammonia (NH₃), NH₄OH, NH₄⁺, and the like.

In one embodiment the composition of the invention may comprise thickener and/or stabilizer, such as pectin (e.g. HM pectin, LM pectin), gelatin, CMC, Soya Bean Fiber/Soya Bean Polymer, starch, modified starch, carrageenan, alginate, and guar gum

In one embodiment wherein the microorganism produces a polysaccharide (such as EPS) which causes a high/ropy texture in the acidified milk product the acidified milk product is produced substantially free, or completely free of any addition of thickener and/or stabilizer, such as pectin (e.g. HM pectin, LM pectin), gelatin, CMC, Soya Bean Fiber/Soya Bean Polymer, starch, modified starch, carrageenan, alginate, and guar gum. By substantially free should be understood that the product comprises from 0% to 20% (w/w) (e.g. from 0% to 10%, from 0% to 5% or from 0% to 2% or from 0% to 1%) thickener and/or stabilizer.

As disclosed previously the interplay between microbial species is crucial-on only in terms of growth but equally is the rheological properties (such as texture) and the development of taste.

### Method of Producing a Fermented Product

One aspect of the invention relates to a method for producing a fermented food product comprising adding a starter culture composition as described herein to a food product starting material and keeping the thus inoculated starting material under conditions where the lactic acid bacterium is metabolically active.

Useful food product starting materials i.e. substrates include any material which is conventionally subjected to a lactic acid bacterial fermentation such as milk, vegetable materials, meat products, fruit juices, must, doughs and batters.

The fermented products, which are obtained by the method, include as typical examples dairy products such as cheese including fresh cheese products, and buttermilk.

Yet another significant application of the lactic acid bacterium according to the present invention is the use of the bacterial cultures as so-called probiotics. By the term “probiotic” is in the present context understood a microbial culture which, when ingested in the form of viable cells by humans or animals, confers an improved health condition, e. g. by suppressing harmful microorganisms in the gastrointestinal tract, by enhancing the immune system or by contributing to the digestion of nutrients.

The term “milk” is to be understood as the lacteal secretion obtained by milking any mammal, such as cows, sheep, goats, buffaloes or camels. In a preferred embodiment, the milk is cow's milk. The term milk also includes protein/fat solutions made of plant materials. Suitable plant material may be legumes, nuts or cereals. Preferred legumes include soybeans, peas, beans, lupins, and lentils. Preferred nuts include coconut, almond, cash, and walnut. Preferred cereals include oat, wheat, rye and millet. Milk may be derived from mammals, from plant materials, or any combination thereof.

The term “milk substrate” may be any raw and/or processed milk material that can be subjected to fermentation according to the method of the invention. Thus, useful milk substrates include, but are not limited to, solutions/suspensions of any milk or milk like products comprising protein, such as whole or low fat milk, skim milk, buttermilk, reconstituted milk powder, condensed milk, dried milk, whey, whey permeate, lactose, mother liquid from crystallization of lactose, whey protein concentrate, or cream. Obviously, the milk substrate may originate from any mammal, e.g. being substantially pure mammalian milk, or reconstituted milk powder or the milk substrate may originate wholly or partly from a plant material i.e. a plant milk as described supra. Preferably, at least part of the protein in the milk substrate is (i) proteins naturally occurring in mammalian milk, such as casein or whey protein or (ii) proteins naturally occurring in plant milk. However, part of the protein may be proteins which are not naturally occurring in milk.

Prior to fermentation, the milk substrate may be homogenized and pasteurized according to methods known in the art.

“Homogenizing” as used herein means intensive mixing to obtain a soluble suspension or emulsion. If homogenization is performed prior to fermentation, it may be performed so as to break up the milk fat into smaller sizes so that it no longer separates from the milk. This may be accomplished by forcing the milk at high pressure through small orifices.

“Pasteurizing” as used herein means treatment of the milk substrate to reduce or eliminate the presence of live organisms, such as microorganisms. Preferably, pasteurization is attained by maintaining a specified temperature for a specified period of time. The specified temperature is usually attained by heating. The temperature and duration may be selected in order to kill or inactivate certain bacteria, such as harmful bacteria. A rapid cooling step may follow.

“Fermentation” in the methods of the present invention means the conversion of carbohydrates into alcohols or acids through the action of a microorganism. Preferably, fermentation in the methods of the invention comprises conversion of lactose to lactic acid.

Fermentation processes to be used in production of fermented milk products are well known and the person of skill in the art will know how to select suitable process conditions, such as temperature, oxygen, amount and characteristics of microorganism(s) and process time. Obviously, fermentation conditions are selected so as to support the achievement of the present invention, i.e. to obtain a fermented product such as a dairy or dairy analogue product in solid or liquid form (fermented milk product).

In the present context the term “starter culture” is a culture which is a preparation (composition) of one or more bacterial strains (such as lactic acid bacteria strains) to assist the beginning of the fermentation process in preparation of fermented products such as various foods, feeds and beverages.

In the present context, a “yoghurt starter culture” is a bacterial culture which comprises one or more Lactobacillus selected from a L. bulgaricus strain and/or an L. acidophilus strain and one or more S. thermophilus strains. In accordance herewith, a “yoghurt” refers to a fermented milk product obtainable by inoculating and fermenting a milk substrate with a composition comprising a Lactobacillus strain such as L. bulgaricus and/or L. acidophilus and a S. thermophilus strain.

It may be preferred that the substrate is a milk substrate. The milk substrate may be derived from an animal, preferably a mammalian, or from a plant. However, the milk substrate need not be purely animal derived, it may further comprise a plant derived substrate. The fermented product may be a food product which again may be a dairy product.

Depending on the product to be produced the substrate may be a milk substrate. A milk substrate is particularly preferred when fermented milk products such as yoghurt, buttermilk or kefir is the final product.

The milk substrate may be an animal or plant derived product. Thus, in an embodiment the fermented product is a food product such as a dairy product. The dairy product may be selected from the group consisting of a fermented milk product such as but not limited to yoghurt, buttermilk and kefir or cheese such as but not limited to fresh cheese or pasta filata.

Likewise, an enzyme may be added to the substrate e.g. the milk substrate before, during and/or after the fermenting, the enzyme being selected from the group consisting of an enzyme able to crosslink proteins, transglutaminase, an aspartic protease, lactase, chymosin, rennet and mixtures thereof.

In one aspect the invention relates to a method for producing a fermented food product, comprising fermenting a substrate with the lactic acid bacterium of the invention, or a composition of the invention.

### Fermented Product

The term “fermented milk product” as used herein refers to a food or feed product wherein the preparation of the food or feed product involves fermentation of a milk substrate with lactic acid bacteria. “Fermented milk product” as used herein includes but is not limited to products such as yogurt and cheese. Other examples of fermented products may be e.g. buttermilk, kefir, quark, tvorog, crème fraiche, and sour cream.

In one embodiment the fermented food product of the invention, wherein the product is yogurt, buttermilk, kefir, quark, tvorog, crème fraiche, sour cream or cheese. Preferably, the fermented milk product is a yogurt.

Examples of cheeses which are prepared by fermentation with S. thermophilus and Lactobacillus delbrueckii subsp. bulgaricus include Mozzarella and pizza cheese (Hoier et al. (2010) in The Technology of Cheesemaking, 2^{nd} Ed. Blackwell Publishing, Oxford; 166-192).

The term “stirred type product” specifically refers to a fermented milk product which sustains a mechanical treatment after fermentation, resulting in a destructuration and liquefaction of the coagulum formed under the fermentation stage. The mechanical treatment is typically but not exclusively obtained by stirring, pumping, filtrating or homogenizing the gel, or by mixing it with other ingredients. Stirred type products typically but not exclusively have a milk solid non-fat content of 9-15%.

The term “set-type product” includes a product based on milk which has been inoculated with a starter culture, e.g. a starter culture, and packaged next to the inoculating step and then fermented in the package.

In one embodiment the fermented product may be in the form of a stirred type product, a set type product or a drinkable product.

Even though the fermented product and/or the food product itself comprise acid and flavor generated during fermentation it may be desired that fermented product and/or the dairy product comprises an ingredient selected from the group consisting of a fruit concentrate, a syrup, a probiotic bacterial strain or culture, a coloring agent, a thickening agent, a flavoring agent, a preserving agent and mixtures thereof. In one embodiment of the invention the fermented food product comprises an ingredient selected from the group consisting of: a fruit concentrate, a syrup, a probiotic bacterial strain or culture, a coloring agent, a thickening agent, a flavoring agent, a preserving agent and mixtures thereof.

It will be appreciated that aspects and embodiments disclosed in the parts termed “Phage-resistant lactic acid bacteria”, “Composition”, “Method of producing a fermented product” and “Fermented Product” may each be equally relevant to the other parts of the specification. Especially disclosures about specific strain(s) of the present invention are applicable to all parts of the specification which is clear for the person skilled in the art.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The listing or discussion of an apparently prior published document in this specification should not necessarily be taken as an acknowledgement that the document is part of the state of the art or is common general knowledge.

Preferences, options and embodiments for a given aspect, feature or parameter of the invention should, unless the context indicates otherwise, be regarded as having been disclosed in combination with any and all preferences, options and embodiments for all other aspects, features and parameters of the invention. This is especially true for the description of the fat encapsulated microbial culture and all its features, which may readily be part of the final composition or product obtained by the method as described herein. Embodiments and features of the present invention are also outlined in the following items.

### Deposits and Expert Solution

The applicant requests that a sample of the deposited microorganisms stated in the table below may only be made available to an expert, until the date on which the patent is granted.

TABLE 2
Deposits made at a Depositary institution having acquired the
status of international depositary authority under the Budapest
Treaty on the International Recognition of the Deposit of
Microorganisms for the Purposes of Patent Procedure: Leibniz
Institute DSMZ-German Collection of Microorganisms and
Cell Cultures Inhoffenstr. 7B, 38124 Braunschweig, Germany.

| Deposited Microorganism | Accession No. | Deposit date |
|---|---|---|
| Streptococcus thermophilus | DSM 34236 | 2022 Apr. 7 |
| Streptococcus thermophilus | DSM 34235 | 2022 Apr. 7 |
| pac type bacteriophage | DSM 34256 | 2022 Apr. 7 |
| cos type bacteriophage | DSM 34257 | 2022 Apr. 7 |

### References

All references cited in this patent document are hereby incorporated herein in their entirety by reference.
Hoier et al. (2010) The Technology of Cheesemaking, 2^{nd} Ed. Blackwell Publishing, Oxford, p.166-192.
Zheng et al. (2020 Apr. 15) Int J Syst Evol Microbiol. Vol.70(4), p.2782-2858.

### EXAMPLES

### Example 1—Preparation of a Phage-Resistant Mutant

### Isolation of Phage-Resistant Strain

Phage-resistant mutants were generated from the mother strain DSM 34236 on M17-2% lactose agar plates with 10 mM MgCl₂/CaCl₂ after plating 0.1ml of an M17-2% lactose overnight culture of DSM 34236 together with 0.1 mL of phage DSM 34256 containing 1×10⁹ phage particles per mL and incubation overnight at 37° C. For this, phages and host strain were mixed with 2.5 mL of a top agar solution (molten agar with half (0.75%) of the standard agar concentration kept at 46-50° C.) and poured on a bottom M17-2% lactose agar. Both bottom and top agar comprising 10 mM MgCl₂/CaCl². When the top agar was solidified incubation occurred with conditions described above.

Among a number of phage-resistant mutants which appeared on the agar plates after incubation, the strain DSM 34235 was three times colony purified and retested in plaque assay on M17 lactose agar plates at 37° C. test using phage DSM 34256 for phage challenge.

Phage-resistance towards DSM 34256 was confirmed by no single plaques observed in plaque test. Phage-resistance towards phage DSM 34257 was also observed.

### Example 2—Viscosity of a Phage-Resistant Mutant

### Pipette Testing

The phage-resistant mutants of the invention were further selected based on viscosity. Viscosity was measured by pipette test for a number of purified phage-resistant mutants. In this test, the efflux time from a volumetric pipette is determined. A longer efflux time corresponds to higher viscosity. Coagulated milk was made from 200 mL skimmed milk inoculated with 1% of the bacterial strains to be tested (from an overnight culture grown in skimmed milk at 37° C.), and incubated for 20 h at 37° C. The viscosity of the coagulated milk was measured with a 25 mL volumetric pipette where the efflux time of said coagulated milk from the pipette was measured in triplicates. The coagulated milk is stirred carefully with a spoon to homogenize. The 25 mL volumetric pipette is then filled and the time to empty the pipette by gravity force is measured. The time it takes to empty 25 mL of coagulated milk from the pipette is noted as seconds.

DSM 34235 provides a higher viscosity than its mother strain DSM 34236 as it yields a 15% increase in efflux time measured by pipette test as shown in the table below.

TABLE 3
Viscosity of DSM 34235, its mother strain DSM 34236 and a prior art strain.

| | Efflux time | Efflux time | Efflux time | Average | |
|---|---|---|---|---|---|
| | sample 1 | sample 2 | sample 3 | efflux time | Standard |
| | (s) | (s) | (s) | (s) | deviation |
| DSM 22589 | 31 | 31 | 32 | 31 | 0.58 |
| DSM 34236 | 37 | 43 | 37 | 39 | 3.46 |
| DSM 34235 | 44 | 44 | 48 | 45 | 2.31 |

In another experiment DSM 34235 showed a 39% increase in efflux time measured by pipette test in comparison with DSM 22589 as apparent in the table below.

TABLE 4
Viscosity of DSM 34235 and a prior art strain.

| | Efflux time | Efflux time | Efflux time | Average | |
|---|---|---|---|---|---|
| | sample 1 | sample 2 | sample 3 | efflux time | Standard |
| | (s) | (s) | (s) | (s) | deviation |
| DSM 22589 | 35 | 44 | 30 | 36 | 7.09 |
| DSM 34235 | 53 | 51 | 45 | 50 | 4.16 |

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Example 3—Acidification Profile of a Phage-Resistant Mutant

### Acidification Assay

For the acidification test 200 mL skimmed milk was inoculated with 1% of the bacterial strains to be tested (from an overnight culture grown in M17-2% lactose incubated at 37° C.), and incubated for 18 h at 37° C. The pH was followed over time with the CINAC system (Scientific Solutions).

The acidification profile of the strain DSM 34235 was similar to the acidification profile of its mother strain indicating that the acidification activity of the strain DSM 34235 is comparable with the acidification activity of its mother strain.

1. A lactic acid bacterium with increased phage-resistance towards DSM 34256 and/or DSM 34257 as compared to its mother strain.

2. The lactic acid bacterium of claim 1, wherein the bacterium has increased viscosity as compared to its mother strain.

3. The lactic acid bacterium of claim 1, wherein the bacterium belongs to the species Streptococcus thermophilus.

4. The lactic acid bacterium of claim 1, wherein the mother strain is DSM 34236.

5. The lactic acid bacterium of claim 1, wherein the bacterium is selected from the group consisting of: DSM 34235, and mutants and variants thereof.

6. A composition comprising the lactic acid bacteria of claim 1.

7. The composition of claim 6, further comprising, either as a mixture or as a kit-of-parts one or more strains belonging to a Lactobacillus species.

8. The composition of claim 6, wherein at least one Lactobacillus strain is Lactobacillus delbrueckii subsp. bulgaricus.

9. The composition of claim 6, wherein the Lactobacillus bulgaricus strain is selected from the group consisting of: DSM 19251; DSM 19252; DSM 22586; DSM 26419; DSM 28910; DSM 32092; and DSM 33515.

10. The composition of claim 6, comprising either as a mixture or as a kit-of-parts one or more Streptococcus thermophilus strains selected from DSM 17876; DSM 18111;DSM 19242; DSM 21408; DSM 22585; DSM 22587; DSM 22588;
DSM 22589; DSM 22935; DSM 24023; DSM 25012; DSM 26562; DSM 32503; DSM 32826; and
DSM 33869.

11. The composition of claim 6, comprising either as a mixture or as a kit-of-parts one or more probiotic strains selected from Bifidobacterium, Lactobacillus acidophilus, Lacticaseibacillus casei, Lacticaseibacillus paracasei, Lacticaseibacillus rhamnosus, and Limosilactobacillus fermentum.

12. A method for producing a fermented food product, comprising fermenting a substrate with the lactic acid bacterium of claim 1.

13. A fermented food product comprising the lactic acid bacterium of claim 1.

14. The fermented food product of claim 13, wherein the product is a dairy product.

15. The fermented food product of claim 13, wherein the product is yogurt, buttermilk, kefir, quark, tvorog, crème fraiche, sour cream or cheese.

16. The fermented food product of claim 15, wherein the yogurt is in the form of a stirred type product, a set type product, or a drinkable product.
